(19) United States
(12) Patent Application Publication
(10) Pub. No.: US 2012/0322892 A1
(43) Pub. Date: Dec. 20, 2012

(54) ORAL MICROEMULSION OF ELEMENE

(75) Inventors: Tian Xie, Dalian City (CN); Zhaowu Zeng, Dalian City (CN); Guanglin Zhou, Dalian City (CN)

(73) Assignee: Tian XIE, Dalian City, Liaoning Province (CN)

(21) Appl. No.: 13/581,059

(22) PCT Filed: Feb. 24, 2011
(86) PCT No.: PCT/CN2011/071240

§ 371 Date: Aug. 24, 2012

(30) Foreign Application Priority Data
Feb. 25, 2010 (CN) 201010114096.6

(51) Int. Cl. A61K 31/015; A61P 35/00

(52) U.S. Cl. 514/763

(57) ABSTRACT

An oral microemulsion of elemene is disclosed. The microemulsion is made of elemene, surfactant, cosurfactant and water or buffer solutions with pH 5˜8. Said surfactant is selected form tweens, polyoxyethylene castor oils, polyethylene glycol stearates and mixture thereof. Said cosurfactant is selected from ethanol, 1,3-propanediol, glycerol and mixture thereof. The concentration of elemene in the microemulsion is 1˜5 g/100 ml. The ratio of elemene, surfactant and cosurfactant is 1˜5(weight):1˜40(weight):1˜40(volume), wherein the unit of weight/volume is g/ml.

### FIELD OF THE INVENTION

The present invention is directed to the field of medicine technology, and relates to an anti-tumor medicament, the elemene microemulsion formulation.

### BACKGROUND

Elemene is an anti-cancer agent extracted from a Chinese medicine and has been marketed for more than 10 years, with an established efficacy and mild toxic and side effects. It has been shown to be an active ingredient with anti-cancer activity, extracted from Wen Yu Jin (Curcuma wenyujin), one of the traditional Chinese medicine “Zhe Ba Wei (Eight Herbs in Zhejiang)”. Elemene belongs to sesquiterpene compounds consisting of two elements, carbon and hydrogen. The primary ingredient in the elemene is β-elemene, and there are also γ- and δ-elemenes. The Wen Yu Jin, also known as wenchow turmeric rhizome, is defined as the dried rhizomes of Curcuma wenyujin Y. H. Chen et C. Ling, in the Pharmacopoeia of People's Republic of China (First Section).

The elemene is a non-cytotoxic anti-cancer agent extracted from a Chinese medicine with an established efficacy and good anti-cancer activities. The elemene have mild toxic and side effects, imposes no significant impairments on cardiac, hepatic and renal functions, and does not cause bone marrow suppression, as compared to chemotherapeutic agents with cytotoxicity. However, it has somewhat irritation and side-effects, which manifest primarily as: (1) phlebitis; (2) pyrexia; and (3) irritant chest or abdominal pains. Two types of dosage forms for the elemene exist: elemene injections and oral liquids. These formulations are primarily administrated via intravenous dripping or oral administration, which are both conventional dosage form and classic route of administration. Elemene is an drug with lipid solubility and hardly soluble in water. The existing oral elemene formulation has a significant first-pass effect and thus less bioavailability. There is great need for resolving the disadvantage in water-solubility of the elemene, that is, increasing its bioavailability while reducing its side-effects.

Microemulsion is an isotropic, transparent, and thermodynamically stable dispersion system formed spontaneously upon mixing water, an oil, a surfactant and a co-surfactant in a proper ratio. In addition to typical features of an emulsion, microemulsion has special advantages such as small particle diameter, transparency, stable, etc., which also enables its wide usage in the pharmaceutical formulation and the clinical context. At present, microemulsion is an novel carrier ideal for drug release. It possesses characteristics such as transparency, being stable, high bioavailability, targeted delivery etc. by which the efficacy of a drug will be increased and its toxic and side effects may be reduced. With the increasing value in clinical application, microemulsion will have a great prospect. Generally, the particle diameter distribution for typical emulsion droplets (namely, an interval between the minimal and the maximal values for the particle diameter) is from 0.1 to 10 μm, thereby forming an opaque milk-white liquid. If the particle diameter distribution for the droplets is between 0.1 and 1.5 μm, the emulsion for intravenous injection is a sub-microemulsion; if the distribution is from 0.01 to 0.1 μm (that is, 10-100 nm), the emulsion is a microemulsion or micellar emulsion, when the particle diameter of the emulsion falls within that of a colloid dispersion, thus forming a transparent or semi-transparent liquid.

If elemene can be formulated into the dosage form of an oral microemulsion, a path way for overcoming the disadvantages that the elemene has low bioavailability in clinical context and low water-insolubility may be provided, for the dosage form enables more stable blood concentration, reduced side-effects, and increased patient's compliance. Currently, however, there has no successful reports about formulating the elemene into the dosage form of oral microemulsion.

### SUMMARY OF THE INVENTION

An object of the invention is to provide an anti-tumor plant-medicament elemene oral microemulsion for purpose of increasing the bioavailability of the elemene and reducing its side-effects.

To achieve the above-mentioned object of the invention, a technical scheme was adopted in the present invention as follows:

An oral microemulsion anti-tumor plant-medicament elemene is made from raw material components fed at the following ratio; said raw material components include elemene, a surfactant, a co-surfactant and water or a buffer solution at a pH ranging from 5 to 8; the surfactant is selected from one of or admixture of several ones of the following materials at any ratio: Tweens, polyoxyethylene castor oils, and polyethylene glycol separates surfactants; the co-surfactant is selected from any one of and admixture of several ones of the following materials at any ratio: ethanol, 1,3-propylene glycol, and glycerin; the buffer solution is selected from one of the following: phosphate salt buffer, ethanol-acetic acid buffer, Tris-hydroxymethyl aminomethane buffer, phthalate buffer, citrate buffer, citric acid-disodium hydrogen phosphate buffer, ammonia-ammonium chloride buffer, acetate salt buffer, acetic acid-sodium acetate buffer, acetic acid-ammonium acetate buffer, phosphoric acid-triethylamine buffer; the pH of the elemene oral microemulsion ranges from 5 to 8; the concentration of elemene in the elemene oral microemulsion ranges from 1 g/100 ml to 5 g/100 ml, wherein the ratio of elemene:surfactant:co-surfactant in the elemene oral microemulsion is 1-5 weight parts:1-40 weight parts:1-40 volume parts. Preferable, the ratio of elemene:surfactant:co-surfactant is 1-5 weight parts:5-40 weight parts:5-40 volume parts. The unit for the volume part/volume part as set forth in the invention is g/ml.

For choosing water and a buffer at a pH ranging from 5 to 8 for use in the elemene oral microemulsion set forth in the present invention, if the pH of the elemene oral microemulsion ranges from 5 to 8 after mixing the elemene, the surfactant, and the co-surfactant together, addition of water may be chose; if the pH does not range from 5 to 8, it needs to be adjusted by adding the buffer at a pH ranging from 5 to 8.

The surfactant set forth in the present invention is selected from one of or admixture of any of several ones of the following surfactants at any ratio: Tweens, polyoxyethylene castor oils, and polyethyleneglycol stearates surfactants, Tween-type surfactants encompass: Tween 80 and Tween 20; The surfactants of polyoxyethylene castor oil include polyoxyethylene castor oils and derivatives thereof; The polyethyleneglycol stearates include polyethyleneglycol stearates and derivatives thereof. It is preferred in the present invention that the surfactant is selected from one of or admixture of any of several ones of the following surfactants at any ratio: Tween 80, polyoxyethylene castor oil, polyethyleneglycol-12-hydroxy stearate, more preferably the admixture of more than two of the following: tween 80, polyoxyethylene castor oil, and polyethyleneglycol 12-hydroxy stearate.

The raw material components set forth in the present invention further comprise an antioxidant, wherein the ratio by weight of the antioxidant to elemene is 0-0.05:1-5 and the lower limit “0” means to approach infinitely but never equal to zero, said antioxidant is selected from one of or admixture of several ones of the following: sodium sulfite, sodium hydrogen-sulfite, sodium pyrosulfite, sodium hyposulfite, propyl gallate, ascorbyl palmitate, t-butyl p-hydroxyanisole, di-t-butyl p-cresol, vitamin E, vitamin C, cysteine, methionine, citric acid, malic acid, sorbitol, ascorbyl palmitate, ethanolamine, and phosphatides. Addition of the antioxidant has little effect on formation of the microemulsion.

The raw material components set forth in the present invention further comprise a preservative, wherein the ratio by weight of the antioxidant to elemene is 0-0.05:1-5 and the lower limit “0” means to approach infinitely but never equal to zero, said preservative is selected from one of the following: parabens, sorbic acid, sorbates, benzoic acid, and benzoates. It is preferable that the preservative is parabens, such as methylparaben, ethyl paraben etc., more preferably ethyl paraben. Addition of a preservative has little effect on formation of the microemulsion.

The preferred embodiment 1 is as follows:

The elemene oral microemulsion is made from elemene, a surfactant, a co-surfactant and water; the charging amounts of the individual raw material components are expressed by the volume of the elemene oral microemulsion as follows:

| | |
|---|---|
| elemene | 1-5 g/100 ml |
| surfactant | 5-40 g/100 ml |
| co-surfactant | 5-40 ml/100 ml |

the balance is water;

said surfactant is selected from one of or combination of any of several ones of the following surfactants at any ratio: Tween 80, polyoxyethylene castor oil, and polyethyleneglycol 12-hydroxy stearate; said co-surfactant is selected from any one of and admixture of several ones of the following materials at any ratio: ethanol, 1,3-propylene glycol, and glycerin.

The preferred embodiment 2 is that:

the elemene oral microemulsion is made from elemene, a surfactant, a co-surfactant, a preservative, and water, the charging amounts of the individual raw material components are expressed by the volume of the elemene oral microemulsion as follows:

| | |
|---|---|
| elemene | 1-5 g/100 ml |
| surfactant | 5-40 g/100 ml |
| co-surfactant | 5-40 ml/100 ml |
| preservative | 0.01-0.05 g/100 ml |

the balance is water;

said surfactant is selected from one of or combination of any of several ones of the following surfactants at any ratio: Tween 80, polyoxyethylene castor oil, and polyethyleneglycol 12-hydroxy stearate; said co-surfactant is selected from any one of and admixture of several ones of the following materials at any ratio: ethanol, 1,3-propylene glycol, and glycerin; the preservative is selected from one of the following: parabens, sorbic acid, sorbates, benzoic acid, and benzoates.

The preferred embodiment 3 is as follows:

the elemene oral microemulsion is made from elemene, a surfactant, a co-surfactant, an antioxidant, a preservative, and water or a buffer at a pH ranging from 5 to 8, the charging amounts of the individual raw material components are expressed by the volume of the elemene oral microemulsion as follows:

| | |
|---|---|
| elemene | 1-5 g/100 ml |
| surfactant | 5-10 g/100 ml |
| co-surfactant | 5-25 ml/100 ml |
| antioxidant | 0.005-0.03 g/100 ml |
| preservative | 0.01-0.05 g/100 ml |

the balance is water or a buffer at a pH ranging from 5 to 8;

said surfactant is selected from one of or combination of any of several ones of the following surfactants at any ratio: Tween 80, polyoxyethylene castor oil, and polyethyleneglycol 12-hydroxy stearate; said co-surfactant is selected from one of or combination of any of several ones of the following surfactants at any ratio: ethanol, 1,3-propylene glycol, and glycerin; said antioxidant is selected from one of and admixture of any of several ones of the following antioxidants: sodium sulfite, sodium hydrogen-sulfite, sodium pyrosulfite, sodium hyposulfite, propyl gallate, ascorbyl palmitate, t-butyl p-hydroxyanisole, di-t-butyl p-cresol, vitamin E, vitamin C, cysteine, methionine, citric acid, malic acid, sorbitol, ascorbyl palmitate, ethanolamine, and phosphatides; the preservative is selected from one of the following: parabens, sorbic acid, sorbates, benzoic acid, and benzoates.

The “water” set forth in the present invention is distilled water or purified water or water for injection.

Furthermore, it is preferred in the present invention that the elemene oral microemulsion is made from elemene, ethanol, glycerin, 1,3-propylene glycol, Tween 80, ethyl paraben and purified water, the charging amounts of the individual raw material components are expressed by the volume of the elemene oral microemulsion as follows: emulsified elemene of 1 g/100 ml, ethanol of 5 ml/100 ml, glycerin of 15 ml/100 ml, 1,3-propylene glycol of 15 ml/100 ml, Tween 80 of 5 g/100 ml, and ethyl paraben of 50 mg/100 ml, the balance is purified water.

The elemene oral microemulsion set forth in the present invention may be prepared by one of or the combination of the more than two methods: agitation, ultrasonication, high-pressure homogenization, and high-velocity homogenization. It is preferable to use of the ultrasonication and high-pressure homogenization. Both the ultrasonication and high-pressure homogenization aid in forming emulsion and reducing the amount of adjuncts used. It is preferable that the ultrasonication comprises ultrasonic dispersion at room temperature in water bath for 1 h with a power for ultrasonication preferably being 400 w. Ultrasonication in water bath may prevent excessively high temperature during the ultrasonication process from having an effect on the quality of the medication. Also the more preferable is the combination of ultrasonication and high-pressure homogenization, that is, ultrasonic dispersion for 10 min (ultrasonicating at room temperature in water bath, with a power of 400 w), followed by performance of high-pressure homogenization once at 600 bar.

It is recommended specifically in the present invention that the elemene oral microemulsion is prepared by the following process: preparing the raw materials in the ratio set forth in the present invention, mixing part of water or a buffer at a pH ranging from 5 to 8 homogenously with the other raw material components, ultrasonicating the resultant mixture at room temperature for 0.1-2 h, allowing the mixture to be cooled to room-temperature, filtering the mixture with a 0.22 μm micro pore filter membrane, adding the remainder of water or a buffer at a pH ranging from 5 to 8 to give the elemene oral microemulsion.

The elemene oral microemulsion can be used in treating a wide variety of malignancies, such as, malignant pleural effusion, malignant seroperitoneum, lung cancers, brain tumors, brain metastatic tumors, respiratory tract tumor, digestive tract tumor, gynecological tumor, mammary cancers, skin cancers, osseous metastasis carcinomas, lymphomas, oral cancer, urologic neoplasms, leukemias. etc.

The advantages of the present invention are as follows: overcoming the disadvantage of the elemene being water-insoluble, increasing the bioavailability, allowing the blood concentration to be more stable, reducing the side-effects in comparison with the existing elemene oral formulations, and enhancing anti-tumor efficacy; convenient oral administration; accurate dosage, which is advantageous for increasing the patient's compliance; saving raw materials and costs, the simplified process, simple preparation and easiness for industrialization, due to direct use of the elemene volatile oil as the oil phase; individual ingredients in the formulation being physiologically compatible substances, safety, and easy to be bought. The elemene oral microemulsion can be prepared by adjusting the amounts of the raw materials in the formula, and the particle diameters can be controlled to be adapted for various routes of administration and dosing requirements.

### BRIEF DESCRIPTION OF THE DRAWINGS

FIG. 1 is the plasma concentration time-curve for the elemene oral microemulsion vs. emulsion obtained from Example 6.

### DETAILED DESCRIPTION OF THE INVENTION

The invention will be illustrated further in conjunction with the specific Examples below. The embodiment herein is intended to be illustrative only and is not to be construed in any way as limiting the scope of the invention.

### Example 1

Formula: elemene 1 g, ethanol 5 ml, glycerin 15 ml, propropylene glycol 1 5 ml, Tween (80) 5 g, ethyl paraben 50 mg, purified water filled up to 100 ml.

preparative process: mixing the prescribed amounts of the elemene, Tween 80, ethyl paraben, ethanol, glycerin, and propropylene glycol to homogeneity, adding 60 ml of water, mixing the resultant mixture homogenously, performing ultrasonication on the resultant mixture at room temperature for 1 h, cooling the resultant mixture to room temperature, filtering the mixture with a 0.22 μm micro pore filter membrane, adjusting the volume of the mixture to 100 ml by addition of purified water, and subpackaging to yield the elemene oral microemulsion. The elemene oral microemulsion thus prepared has a pH of 4, a viscosity of 6 mPa·s, a surface tonicity of 32.1 mN/m, an average particle diameter of 67 nm determined by a laser particle size analyzer (mode: LS230 laser particle size analyzer, manufacturer: Beckman Coulter Co., Ltd., U.S.A.) with a particle diameter range varying from 54 nm to 80 nm.

### Example 2

Formula: elemene 1 g, ethanol 5 ml, glycerin 10 mL, Tween (80) 5 g, polyoxyethylene castor oil (EL) 1.25 g, ethyl paraben 50 mg, purified water filled up to 100 ml.

Preparative process: dissolving the prescribed amounts of the elemene, Tween (80), polyoxyethylene castor oil (EL), and ethyl paraben in ethanol and glycerin, then adding 50 ml of purified water and mixing the resultant mixture homogenously, performing ultrasonication on the resultant mixture at room temperature for 1 h, cooling the resultant mixture to room temperature, filtering the mixture with a 0.22 μm micro pore filter membrane, adjusting the volume of the mixture to 100 ml by addition of water, and subpackaging to yield the elemene oral microemulsion. The elemene oral microemulsion thus prepared has a pH of 5.26, a viscosity of 4 mPa·s, a surface tonicity of 34.7 mN/m, an average particle diameter of 54 nm with a particle diameter range varying from 46.2 nm to 61.8 nm.

### Example 3

Formula: elemene 1 g, ethanol 5 ml, glycerin 10 mL, propropylene glycol 5 mL, Tween (80) 2.5 g, polyoxyethylene castor oil (EL) 3.75 g, vitamin C 25 mg, ethyl paraben 50 mg, 0.1 M phosphate buffered liquid (pH7.0) filled up to 100 ml.

Preparative process: dissolving the prescribed amounts of the elemene, Tween (80), polyoxyethylene castor oil (EL), and ethyl paraben in ethanol, glycerin and propropylene glycol and mixing to homogeneity, dissolving vitamin C in 50 ml of PBS buffer, adding the oil phase into the aqueous phase and mixing homogenously, performing ultrasonication on the resultant mixture at room temperature for 1 h, cooling the resultant mixture to room temperature, filtering the mixture with a 0.22 μm micro pore filter membrane, adjusting the volume of the mixture to 100 ml by addition of the buffer, and subpackaging to yield the elemene oral microemulsion. The elemene oral microemulsion thus prepared has a pH of 6.84, a viscosity of 5 mPa·s, a surface tonicity of 35.6 mN/m, an average particle diameter of 60 nm with a particle diameter range varying from 50.8 nm to 69.2 nm.

### Example 4

Formula: elemene 5 g, polyethyleneglycol-12-hydroxy stearate (Solutol HS15) 3 g, ethanol 40 ml, Tween (80) 30 g, pure water filled up to 100 ml.

Preparative process: dissolving the prescribed amounts of the elemene, Tween (80), Solutol HS15 in ethanol and mixing homogenously the resultant mixture, adding water to 80 ml and mixing the resultant mixture homogenously, performing ultrasonication on the resultant mixture at room temperature for 1 h, cooling the resultant mixture to room temperature, filtering the mixture with a 0.22 μm micro pore filter membrane, adjusting the volume of the mixture to 100 ml by addition of purified water, and subpackaging to yield the elemene oral microemulsion. The elemene oral microemulsion thus prepared has a pH of 6.35, a viscosity of 64 mPa·s, a surface tonicity of 30.0 mN/m, an average particle diameter of 72 nm with a particle diameter range varying from 57 nm to 87 nm.

### Example 5

Formula: elemene 1 g, ethanol 20 ml, Solutol (HS15) 5 g, and pure water filled up to 100 ml.

Preparative process: dissolving the prescribed amounts of the elemene and Solutol HS15 in ethanol and mixing homogenously the resultant mixture, adding water to 80 ml and mixing the resultant mixture homogenously, performing ultrasonication on the resultant mixture at room temperature for 1 h, cooling the resultant mixture to room temperature, filtering the mixture with a 0.22 μm micro pore filter membrane, adjusting the volume of the mixture to 100 ml by addition of purified water, and subpackaging to yield the elemene oral microemulsion. The elemene oral microemulsion thus prepared has a pH of 5.43, a viscosity of 4 mPa·s, a surface tonicity of 32.8 mN/m, an average particle diameter of 64 nm with a particle diameter range varying from 53 nm to 75 nm.

### Example 6

### Test of the Elemene Oral Microemulsion on the Relative Bioavailability

1.1 Dosing in the Animals and Treatment of a Blood Sample

90 of SD rats having body weight of 140-200 g were selected without limitation on male or female, randomized into the elemene emulsion group (the elemene oral emulsion from Dalian Holley Kingkong Pharmaceutical Co., Ltd., specification 0.2 g/20 mL, lot No. 0904231) and the elemene oral microemulsion group (Example 1, lot No. 09111901), and orally administrated with the microemulsion or the emulsion at a dose of 100 mg/kg. 3-5 mL of blood samples were collected respectively 0, 0.5, 3, 4, 6, 8, 10, 12, 14, 18, and 24 h post administration and centrifuged at 2000×g. The plasma was collected and stored until use. 0.5 mL of plasma was took and added into 1 mL of acetonitrile, shook for 5 min, left to stand for 5 min, and centrifuged at 14000×g. The supernatant was collected, filtered with a 0.22 μm disposable filter and injected for detection.

1.2 The Plasma Concentration-Time Curve and the Relative Bioavailability

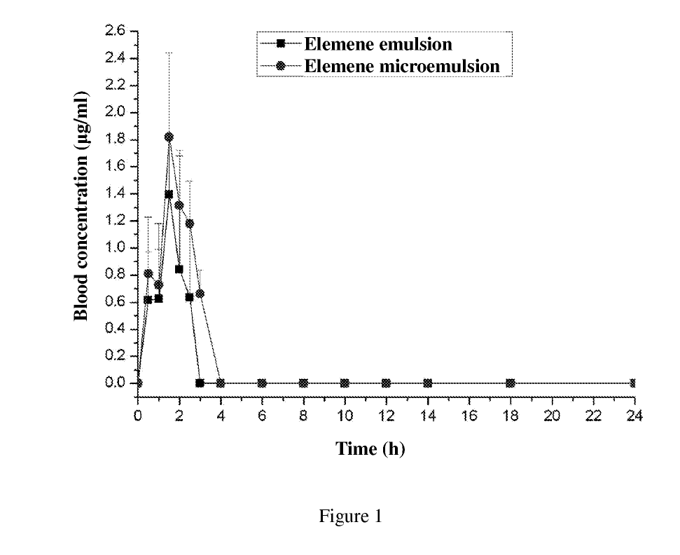

The plasma concentration-time curve for the elemene emulsion vs. microemulsion was plotted (FIG. 1).

The value for area under the curve (AUC) was calculated in a integration way using OriginPro 8.0 software. Since the blood concentrations in the emulsion group and the microemulsion group were zero 24 h post administration, AUC_{0→24 h}=AUC_{0→∞}, AUCₑₘᵤₗₛᵢₒₙ=20.054 μg·h·mL⁻¹; AUCmicroemulsion=3.423 μg·h·mL⁻¹, relative bioavailability F=AUC_{microemulsion}×Dₑₘᵤₗₛᵢₒₙ/AUCₑₘᵤₗₛᵢₒₙ×D_{microemulsion}=166.7%, Dₑₘᵤₗₛᵢₒₙ means dosage of the elemene oral emulsion administrated, D_{microemulsion} means dosage of the elemene oral microemulsion administrated. Cmax_{microemulsion}=1.820 μg·mL⁻¹, Cmaxₑₘᵤₗₛᵢₒₙ=10.395 μg·mL⁻¹. It was found that, the relative bioavailability of the elemene oral microemulsion was more significantly increased to reach 166.7% in comparison with that of the elemene emulsion, and a peak concentration thereof was increased 1.4 times higher.

Disclosed above are merely several specific embodiments of the present invention. However, the present invention should not be limited to these embodiments and any of variations thereof that can be contemplated by those skilled in the art should fall within the claimed scope of the instant invention.

1. An oral microemulsion of anti-tumor plant-medicament elemene made from raw material components fed at the following ratio, characterized in that said raw material components include elemene, a surfactant, a co-surfactant and water or a buffer solution at a pH ranging from 5 to 8;
said surfactant is selected from one of or admixture of any of several ones of the following surfactants at any ratio: Tweens, polyoxyethylene castor oils, and polyethyleneglycol stearates;
said co-surfactant is selected from any one of and admixture of several ones of the following materials at any ratio: ethanol, 1,3-propylene glycol, and glycerin;
said buffer solution is selected from one of the following: phosphate salt buffer, ethanol-acetic acid buffer, Tris-hydroxymethyl aminomethane buffer, phthalate buffer, citrate buffer, citric acid-disodium hydrogen phosphate buffer, ammonia-ammonium chloride buffer, acetate salt buffer, acetic acid-sodium acetate buffer, acetic acid-ammonium acetate buffer, phosphoric acid-triethylamine buffer;
the pH of the elemene oral microemulsion ranges from 5 to 8;
the concentration of elemene in said elemene oral microemulsion ranges from 1 g/100 ml to 5 g/100 ml,
the ratio of elemene:surfactant:co-surfactant in the elemene oral microemulsion is 1-5 weight parts:1-40 weight parts:1-40 volume parts, wherein the unit for the volume part/volume part is g/ml.

2. The elemene oral microemulsion as claimed in claim 1, wherein said surfactant is selected from one of or admixture of several ones of the following surfactants at any ratio: tween 80, polyoxyethylene castor oil, and polyethyleneglycol 12-hydroxy stearate.

3. The elemene oral microemulsion as claimed in claim 1, wherein said raw material components further comprise an antioxidant, wherein the ratio by weight of the antioxidant to elemene is 0-0.05:1-5; said antioxidant selected from one of or admixture of several ones of the following: sodium sulfite, sodium hydrogen-sulfite, sodium pyrosulfite, sodium hyposulfite, propyl gallate, ascorbyl palmitate, t-butyl p-hydroxyanisole, di-t-butyl p-cresol, vitamin E, vitamin C, cysteine, methionine, citric acid, malic acid, sorbitol, ethanolamine, and phosphatides.

4. The elemene oral microemulsion as claimed in claim 1, wherein said raw material components further comprise a preservative, wherein the ratio by weight of the preservative to elemene is 0-0.05:1-5; said preservative is selected from one of the following: parabens, sorbic acid, sorbates, benzoic acid, and benzoates.

5. The elemene oral microemulsion as claimed in claim 1, wherein the elemene oral microemulsion is made from elemene, a surfactant, a co-surfactant and water, the charging amounts of the individual raw material components are expressed by the volume of the elemene oral microemulsion as follows:

| | |
|---|---|
| elemene | 1-5 g/100 ml |
| surfactant | 5-40 g/100 ml |
| co-surfactant | 5-40 ml/100 ml |

the balance is water;
said surfactant is selected from one of or combination of any of several ones of the following surfactants at any ratio: Tween 80, polyoxyethylene castor oil, and polyethyleneglycol 12-hydroxy stearate;
said co-surfactant is selected from any one of and admixture of several ones of the following materials at any ratio: ethanol, 1,3-propylene glycol, and glycerin.

6. The elemene oral microemulsion as claimed in claim 1, wherein said elemene oral microemulsion is made from elemene, a surfactant, a co-surfactant, a preservative, and water, the charging amounts of the individual raw material components are expressed by the volume of the elemene oral microemulsion as follows:

| | |
|---|---|
| elemene | 1-5 g/100 ml |
| surfactant | 5-40 g/100 ml |
| co-surfactant | 5-40 ml/100 ml |
| preservative | 0.01-0.05 g/100 ml |

the balance is water;
said surfactant is selected from one of or combination of any of several ones of the following surfactants at any ratio: Tween 80, polyoxyethylene castor oil, and polyethyleneglycol 12-hydroxy stearate;
said co-surfactant is selected from any one of and admixture of several ones of the following materials at any ratio: ethanol, 1,3-propylene glycol, and glycerin; the preservative is selected from one of the following: parabens, sorbic acid, sorbates, benzoic acid, and benzoates.

7. The elemene oral microemulsion as claimed in claim 1, wherein said elemene oral microemulsion is made from elemene, a surfactant, a co-surfactant, an antioxidant, a preservative, and water or a buffer at a pH ranging from 5 to 8, the charging amounts of the individual raw material components are expressed by the volume of the elemene oral microemulsion as follows:

| | |
|---|---|
| elemene | 1-5 g/100 ml |
| surfactant | 5-10 g/100 ml |
| co-surfactant | 5-25 ml/100 ml |
| antioxidant | 0.005-0.03 g/100 ml |
| preservative | 0.01-0.05 g/100 ml |

the balance is water or a buffer at a pH ranging from 5 to 8;
said surfactant is selected from one of or combination of any of several ones of the following surfactants at any ratio: Tween 80, polyoxyethylene castor oil, and polyethyleneglycol 12-hydroxy stearate;
said co-surfactant is selected from one of or combination of any of several ones of the following surfactants at any ratio: ethanol, 1,3-propylene glycol, and glycerin;
said antioxidant is selected from one of and admixture of any of several ones of the following antioxidants: sodium sulfite, sodium hydrogen-sulfite, sodium pyrosulfite, sodium hyposulfite, propyl gallate, ascorbyl palmitate, t-butyl p-hydroxyanisole, di-t-butyl p-cresol, vitamin E, vitamin C, cysteine, methionine, citric acid, malic acid, sorbitol, ascorbyl palmitate, ethanolamine, and phosphatides; the preservative is selected from one of the following: parabens, sorbic acid, sorbates, benzoic acid, and benzoates.

8. The elemene oral microemulsion as claimed in claim 1, wherein the elemene oral microemulsion is made from elemene, elemene, ethanol, glycerin, 1,3-propylene glycol, Tween 80, ethyl paraben and purified water, the charging amounts of the individual raw material components are expressed by the volume of the elemene oral microemulsion as follows: elemene of 1 g/100 ml, ethanol of 5 ml/100 ml, glycerin of 15 ml/100 ml, 1,3-propylene glycol of 15 ml/100 ml, Tween 80 of 5 g/100 ml, and ethyl paraben of 50 mg/100 ml, the balance is purified water.

9. The elemene oral microemulsion as claimed in claim 1, wherein said elemene oral microemulsion is prepared by one of or the combination of the two methods: ultrasonication and high-pressure homogenization.

10. The elemene oral microemulsion as claimed in claim 9, wherein said elemene oral microemulsion is prepared by the following process: preparing the raw materials in the ratio set forth in any of claims 1-8, mixing part of water or a buffer at a pH ranging from 5 to 8 homogenously with the other raw material components, ultrasonicating the resultant mixture at room temperature for 0.1-2 h, allowing the mixture to be cooled to room-temperature, filtering the mixture with a 0.22 μm micro pore filter membrane, adding the remainder of water or a buffer at a pH ranging from 5 to 8 to give the elemene oral microemulsion.
